(19) United States
(12) Patent Application Publication
(10) Pub. No.: US 2020/0239938 A1
(43) Pub. Date: Jul. 30, 2020

(54) IN-PROCESS METHOD FOR GUIDING MEASURES AGAINST THE PROPAGATION OF SALMONELLA AND/OR AGAINST THE PROPAGATION OF CAMPYLOBACTER IN AN ANIMAL FLOCK

(71) Applicant: EVONIK OPERATIONS GMBH, Essen (DE)

(72) Inventors: Andreas KAPPEL, Glashütten (DE); Florian BÖHL, Neckargemünd (DE); Walter PFEFFERLE, Langgöns (DE); Stefan PELZER, Gütersloh (DE); Emeka Ignatius IGWE, München (DE); Frank THIEMANN, Nottuln (DE)

(73) Assignee: EVONIK OPERATIONS GMBH, Essen (DE)

(21) Appl. No.: 16/652,657

(22) PCT Filed: Sep. 28, 2018
(86) PCT No.: PCT/EP2018/076393

§ 371 Date: Mar. 31, 2020

(30) Foreign Application Priority Data
Oct. 2, 2017 (EP) PCT/EP2017/074985

(51) Int. Cl. C12Q 1/689

(52) U.S. Cl. CPC C12Q 1/689; C12Q 2600/158

(57) ABSTRACT

The present invention relates to an in vitro method for guiding measures against the propagation of Salmonella and/or against the propagation Campylobacter in an animal flock, the method comprising i.) determining the amount of at least one marker gene being specific for Salmonella or Campylobacter in a test sample and ii.) comparing the amount of said at least one marker gene being specific for Salmonella or Campylobacter determined in the test sample with a control sample, wherein an increase in the amount of said at least one marker gene being specific for Salmonella or Campylobacter in the test sample vs. the control sample by at least a factor of two, indicates the necessity of initiating or enhancing measures against the propagation of Salmonella and/or against the propagation Campylobacter.

### FIELD OF THE INVENTION

The present invention relates to a method for guiding measures against the propagation of Salmonella and/or against the propagation of Campylobacter in an animal flock.

### BACKGROUND OF THE INVENTION

Different species within the genus Campylobacter have emerged over the last decades as important clinical pathogens of human and veterinary concern. Campylobacter is a gram-negative bacterium that is a major etiologic agent for acute gastrointestinal diseases in humans. The most common non-specific symptoms are bloody diarrhea, abdominal cramps, fever and vomiting [Black et al. (1988), “Experimental Campylobacter jejuni infection in humans”, J. Infect. Dis. 154:472-479].

Campylobacter bacteria prevail in the gastrointestinal tract of mammals and birds. In chicken, Campylobacter jejuni is the most occurring Campylobacter species. Their intestinal colonization represents the most important source for the contamination of carcasses, meaning that for humans, poultry meat and poultry products, respectively, constitute the major infection sources [Osterom et al. (1983) “Origin and prevalence of Campylobacter jejuni in poultry processing”, J. Food. Prot. 46, 339-344].

Salmonella bacteria are an important cause of food poisoning of humans, which often is linked to the consumption of meat, such as poultry meat, pork or products derived therefrom. Accordingly, controlling Salmonella is a significant challenge for the meat-producing industry. In Europe, a baseline study conducted in 2005 on the prevalence of Salmonella in egg-laying flocks has shown that at the global EU-level, 20.3% of the large-scale laying hen holdings are bacteriologically positive for S. enteritidis. In some countries, the prevalence was higher than 80% [European Food Safety Authority (2006), “Preliminary report: analysis of the baseline study on the prevalence of salmonella in laying hen flocks of Gallus gallus”].

In addition to the above food chain safety risks, Salmonella and/or Campylobacter infections in animals regularly cause considerable economic losses. For example, Salmonella pullorum causes pullorum disease and Salmonella gallinarum causes fowl typhoid.

Laying hens, broilers and turkeys take in these pathogens from their environment at a relatively early age. Only with an excellent hygiene, it is possible to postpone the Campylobacter and/or Salmonella infection. Therefore, the probability of Campylobacter and/or Salmonella colonization increases with animal age and duration of the production/fattening process.

The fast spreading tendency of Campylobacter and Salmonella is attributable to the high bacterial counts in the intestine. Due to the low minimum infective dose, the infection of only one individual animal will result in a spread of the pathogen via fecal contamination of food, bedding material and especially drinking water.

For these reasons, measures aiming at reducing the Campylobacter and/or Salmonella load in an animal flock are to be taken already at an early stage of the production process. Such measures, however, are more or less unspecific. In general, anti-microbial measures in livestock animal production are necessary for maintaining the health of the animals and, at the same time, to ensure economic production. The most efficient anti-microbial measure is using antibiotic agents as feed supplement. However, the emergence and spread of antibiotic resistance have created a growing global threat. Because the use of antibiotics in any setting drives resistance expansion everywhere, it is important to minimize the use of these drugs. Accordingly, inappropriate or non-necessary uses of antibiotic agents should be avoided.

Conventionally, contamination with Campylobacter and/or Salmonella is determined only after slaughter, i.e. samples deriving from individualized animals are analyzed. That is, the success of the above-mentioned anti-microbial measures is only validated or verified after slaughter (post mortem), whereas during the production process, short-term effects resulting from the above measures are not recorded at all. Therefore, partial or total failure of the above measures may not be determined and, consequently, additional countermeasures cannot be taken. Appropriate in-process controls indicating whether the applied measure has led to the desired reduction of Campylobacter and/or Salmonella load are not available.

Further, conventional culture methods for the detection of Campylobacter and/or Salmonella from biological samples are rather complex as these organisms are fastidious and slow growing with specific requirements to incubation atmosphere. Therefore, laboratory diagnosis of Campylobacter and/or Salmonella is time consuming and expensive. In addition thereto, the known methods are often dissatisfying in terms of sensitivity and specificity.

It was thus an urgent need to provide a fast and reliable, sensitive and non-invasive in-process (ante mortem) method for guiding measures against Salmonella and/or against Campylobacter in an animal flock, enabling the farmer to reduce the amount of antibiotics used as feed supplement.

### SUMMARY OF THE INVENTION

The first objective of the present invention is to provide an in vitro method for guiding measures against the propagation of Salmonella and/or against the propagation of Campylobacter in an animal flock, the method comprising
i.) determining the amount of at least one marker gene being specific for Salmonella or Campylobacter in a test sample and
ii.) comparing the amount of said at least one marker gene being specific for Salmonella or Campylobacter determined in the test sample with a control sample, wherein an increase in the amount of said at least one marker gene being specific for Salmonella or Campylobacter in the test sample vs. the control sample by at least a factor of two,
indicates the necessity of initiating or enhancing measures against the propagation of Salmonella and/or against the propagation Campylobacter.

In addition, the present invention provides a diagnostic kit comprising primers and optionally comprising probes for detecting a specific biomarker, wherein said biomarker comprises at least one polynucleotide being specific for the species Campylobacter and/or Salmonella.

### DETAILED DESCRIPTION OF THE INVENTION

The present inventors have unexpectedly found that the necessity—and also the intensity—of health-maintaining, health-promoting or therapeutic measures or interventions may be derived from the dynamic behavior of the Salmonella and/or Campylobacter load in an animal flock.

More precisely, the present inventors have unexpectedly found that an increase in the amount of Salmonella and/or Campylobacter biomarkers in a test sample vs. a control sample by at least a factor of two, in particular by at least a factor of three, indicates the necessity of initiating or enhancing measures against the propagation of Salmonella and/or against the propagation of Campylobacter.

Further, the inventors have found that in case that after applying specific measures against the propagation of Salmonella and/or against the propagation of Campylobacter in an animal flock, the amount of Salmonella and/or Campylobacter biomarkers in a test sample vs. a control sample further increases, initiating further measures or enhancing the previously applied measures against the propagation of Salmonella and/or against the propagation of Campylobacter will be necessary.

Accordingly, the present invention provides an in vitro method for guiding measures against the propagation of Salmonella and/or against the propagation of Campylobacter in an animal flock, the method comprising
i.) determining the amount of at least one marker gene being specific for Salmonella or Campylobacter in a test sample and
ii.) comparing the amount of said at least one marker gene being specific for Salmonella or Campylobacter determined in the test sample with a control sample, wherein an increase in the amount of said at least one marker gene being specific for Salmonella or Campylobacter in the test sample vs. the control sample by at least a factor of two,
indicates the necessity of initiating or enhancing measures against the propagation of Salmonella and/or against the propagation Campylobacter.

An “increased” amount or a “decreased” amount is typically a statistically relevant amount. Thus, an “increased” amount may include an increase that is 2, 3, 4, 5, 6, 7, 8, 9, 10, 15, 20, 30, 40, 50, 100, 200, 250, 500, 1000 or more times more than the natural level of Salmonella and/or Campylobacter, including all integers and decimal points in between the above-specified values. Accordingly, an increase may include e.g. an increase that is at least 0.3 log (log₁₀) or at least 0.5 log (log₁₀).

Conversely, a “decreased” amount of may include an increase that is 2, 3, 4, 5, 6, 7, 8, 9, 10, 15, 20, 30, 40, 50, 100, 200, 250, 500, 1000 or more times less than the natural level of Salmonella and/or Campylobacter, including all integers and decimal points in between the above-specified values. Accordingly, a “decrease” may include e.g. a decrease that is at least 0.3 log (log₁₀) or at least 0.5 log (log₁₀).

A “factor of two” is approximately equivalent to 0.3 log, and a “factor of three” is approximately equivalent to 0.5 log (log₁₀).

By using the above method, a farmer or livestock manager is enabled to address Salmonella and/or Campylobacter contaminations in an animal flock in an individualized and specific manner. Thereby, inappropriate or non-necessary uses of antibiotic agents can be avoided.

The control sample may be a sample deriving from a non-contaminated animal flock or a negative control sample.

In a further and particularly preferred embodiment, the control sample is an earlier stage sample of the animal flock to be tested. That is, the test samples are collected and analyzed at consecutive points in time.

In a different embodiment, the control sample is a sample deriving from the predecessor animal flock kept and/or fattened in the same house or pen.

The sample material of the test sample is preferably a pooled sample of an animal flock (“bulk testing”). A “pooled sample” is to be understood as a composite sample obtained from randomly collected separate samples, i.e. fresh samples taken at random from a number of sites in the house or space in which the animal population is kept. The pooled samples reflect the amount of Salmonella and/or Campylobacter biomarkers in the animal flock.

The animal flock preferably is an avian flock. In a particularly preferred embodiment of the present invention the avian flock is poultry, such as chickens, turkeys, ducks and geese. The poultry can be optimized for producing young stock. This type of poultry is also referred to as parent and grandparent animals. Preferred parent and grandparent animals are, accordingly, (grand)parent broilers, (grand)parent ducks, (grand)parent turkeys and (grand)parent geese.

The poultry according to the invention may also be selected from fancy poultry and wild fowl. Preferred fancy poultry or wild fowl are peacocks, pheasants, partridges, guinea fowl, quails, capercailzies, goose, pigeons and swans. Further preferred poultry according to the invention are ostriches and parrots.

Most preferred poultry according to the invention are broilers.

The sample material may be selected from the group consisting of dust samples, litter samples, liquid manure samples, fur samples, feather samples, skin samples, feed samples, bedding samples and samples of bodily excrements and solutions or suspensions thereof. Bodily excrements are urine, fecal or cecal excrements.

In general, the term “litter” is to be understood as a mixture of animal excrements with the bedding material. The term “litter samples” refers to mixed excremental droppings in the pen, cage or slat. These litter samples can, be collected from a population, for example, by using the overshoe method, or using litter grabs at different places in the pen or collecting samples from any litter removal systems. Further, in the context this embodiment, the term “liquid manure samples” refers to mixed excremental samples containing feces and urine.

Boot swabs being sufficiently absorptive to soak up moisture are particularly suitable for collecting pooled avian samples. Tube gauze socks are also acceptable.

In a particularly preferred embodiment, the sample material is feces.

Suitable sample volumes are, for example, 0.1 to 20 ml, in particular 0.2 to 10 ml, preferably 0.5 to 5 ml. Suitable sample masses are, for example 0.1 to 20 g, in particular 0.2 to 10 g, preferably 0.5 to 5 g.

As used in the context of the present invention, the term “specific biomarker” refers to any biomarker being specific for Salmonella and/or for Campylobacter, for species, sub-species or serovars thereof. Suitable specific biomarkers may be selected from the group consisting of polypeptides, proteins and oligo- or polynucleotides.

As used herein, the terms “oligo- or polynucleotides” refer to DNA or RNA. DNA polynucleotides are particularly suitable.

In a particularly preferred embodiment, the “polynucleotides” are marker genes. In general, suitable marker genes are genes being specific for Campylobacter and for Salmonella, respectively.

Accordingly, in a particularly preferred embodiment the at least one specific biomarker is a marker gene being specific for the genus Salmonella and/or species and/or sub-species and/or serovars thereof. In an alternative, but also particularly preferred embodiment, the at least one specific biomarker is a marker gene being specific for the genus Campylobacter and/or species and/or sub-species and/or serovars thereof.

In one aspect the Salmonella is Salmonella enterica or Salmonella enterica ssp. enterica or its serovars Salmonella enteritidis, Salmonella typhimurium, Salmonella heidelberg, Salmonella kentucky, or any combinations thereof. In another aspect, the Campylobacter is Campylobacter jejuni, Campylobacter coli, Campylobacter lari, Campylobacter upsaliensis, or any combinations thereof. In a different aspect, the Salmonella is Salmonella enterica or Salmonella enterica ssp. enterica or its serovars Salmonella enteritidis, Salmonella typhimurium, Salmonella heidelberg, Salmonella kentucky, or any combinations thereof and the Campylobacter is Campylobacter jejuni, Campylobacter coli, Campylobacter lari, Campylobacter upsaliensis, or any combinations thereof.

Diagnostically relevant genes of Campylobacter species Campylobacter jejuni are, for example, the 23S rRNA gene, the 16S rRNA gene, the 5S rRNA gene, cdtA, cdtB, cdtC, gyrB, glyA, cadF, tuf, atpA, wla genes, cjaA, trpC, cja, cjo, hipO, flaA, fusA, lpxA, Cj0414, and mapA. In a particularly preferred embodiment, the Campylobacter jejuni marker gene is selected from lpxA, Cj0414 and mapA. The marker gene Cj0414 is of particular relevance.

TABLE 1
Selected diagnostically relevant genes of Campylobacter jejuni

| Gene | | NCBI | | NCBI Reference | | |
|---|---|---|---|---|---|---|
| Symbol | Synonyms | GeneID | Protein | Sequence | Reference Strain | Location |
| 1.: IpxA | 1: - | 904599 | Acyl-[acyl-carrier-protein]--UDP-N- | NC_002163.1 | Campylobacter jejuni supsp. Jejuni | 253908- |
| | | | acetylglucosamine O-acyltransferase | | NCTC 81116 | 254699 |
| 2.: Cj0414 | 2: - | 904736 | Oxireductase subunit | NC_002163.1 | Campylobacter jejuni supsp. Jejuni | 380937- |
| | | | | | NCTC 11168 | 381665 |
| 3.: mapA | 3: - | 5617465 | Outer membrane lipoprotein | NC_009839.1 | Campylobacter jejuni supsp. Jejuni | 975040- |
| | | | | | NCTC 81116 | 975684 |

Diagnostically relevant genes of Salmonella species Salmonella enterica ssp. enterica are, for example, ompf gene, inva gene, 23S rRNA gene, 16S rRNA gene, 5S rRNA gene, mutS, recA, arac gene, rpob gene, Salmolysin, mdh gene, tuf A,B genes, phop gene, gyrA gene, parc gene, invE gene and prgH. In a particularly preferred embodiment, the Salmonella enterica ssp. enterica marker gene is selected from the group invA, fliC, invE and hilA. The marker gene hilA is of particular relevance.

TABLE 2
Selected diagnostically relevant genes of Salmonella species Salmonella enterica ssp. enterica

| Gene | | NCBI | | NCBI Reference | | |
|---|---|---|---|---|---|---|
| Symbol | Synonyms | GeneID | Protein | Sequence | Reference Strain | Location |
| 1.: invA | 1: - | 1254419 | EscV/YiscV/HrcV family | NC_003197.2 | Salmonella enterica subsp. enterica | 3038407- |
| | | | type III secretion system | | serovar Typhirnurium str. L12, | 3040471 |
| | | | export apparatus protein | | complete genome | |
| 2.: hypothetical | 2: - | 1255584 | Hypothetical protein | NC_003197.2 | Salmonella enterica subsp. enterica | 4267936- |
| Protein | | | | | serovar Typhirnurium str. L12, | 4268338 |
| | | | | | complete genome | |
| 3.: hilA | 3: iagA | 1254399 | transcriptional regulator | NC_003197.2 | Salmonella enterica subsp. enterica | 3019846- |
| | | | | | serovar Typhirnurium str. L12, | 3021524 |
| | | | | | complete genome | |
| 4: invE | 4: - | 1254420 | SepL/TyeA/HrpJ family | NC_003197.2 | Salmonella enterica subsp. enterica | 3040489- |
| | | | type III secretion | | serovar Typhirnurium str. L12, | 3041615 |
| | | | system gatekeeper | | complete genome | |

The marker polynucleotides or marker genes may be isolated from the animal samples prior to quantification. Polynucleotide isolation can, for example, be performed via extraction using the Cetyltrimethylammoniumbromid (CTAB) method or by diverse commercial nucleic acid extraction kits, in which cell lysis is achieved either through chemical lysis and/or by mechanical cell disruption and nucleic acid is captured on silica matrices or on silica-cladded magnetic beads. Commercial extraction kits specialized on fecal material or harsh material are particularly suitable.

The marker genes may be detected and/or quantified by commonly known methods such as sequencing, next-generation sequencing, hybridization or various PCR techniques known in the art. The term “quantification” or related words refer to determining the quantity, mass, or concentration in a unit volume. In a particularly preferred embodiment, the quantity (amount) amount of Salmonella and/or Campylobacter biomarkers contained in the sample material is determined.

In an alternative embodiment, the marker genes contained in the animal sample may be quantified directly, for example via PCR, qPCR, sequencing or hybridization techniques.

As an example, after an initial determination of the amount of said at least one specific biomarker in a test sample, the amount of said at least one specific biomarker may be monitored in test samples collected and analyzed a weekly, daily our hourly manner. In a preferred embodiment, the animal samples are collected and analyzed at consecutive days.

In one embodiment, test samples are taken and analyzed on a daily basis from birth to slaughter.

In a preferred embodiment for poultry, a first test sample is preferably taken and analyzed during the initial growth phase (starter phase, day 5 to day 10), a second test sample is taken and analyzed during the enhanced growth phase (day 11 to day 18) and, optionally, a third test sample is taken and analyzed in a later stage.

In an alternative embodiment, a first test sample is taken and analyzed in the initial grow phase and further test samples are taken and analyzed for example on a daily basis during the enhanced growth phase, optionally until slaughter.

The measures to be taken against the propagation of Salmonella and/or against the propagation of Campylobacter involve feeding or administering health-promoting substances, such as zootechnical feed additives, or therapeutic agents. The term “administering” or related terms includes oral administration. Oral administration may be via drinking water, oral gavage, aerosol spray or animal feed. The term “zootechnical feed additive” refers to any additive used to affect favorably the performance of animals in good health or used to affect favorably the environment. Examples for zootechnical feed additives are digestibility enhancers, i.e. substances which, when fed to animals, increase the digestibility of the diet, through action on target feed materials; gut flora stabilizers; micro-organisms or other chemically defined substances, which, when fed to animals, have a positive effect on the gut flora; or substances which favorably affect the environment. Preferably, the health-promoting substances are selected from the group consisting of probiotic agents, praebiotic agents, botanicals, organic/fatty acids, bacteriophages and bacteriolytic enzymes or any combinations thereof.

Generally, the measures to be taken may be categorized into “soft measures”, “medium measures” and “hard interventions”. As used in the context of the present invention, “soft measures” involve mainly general hygiene management/hygienic measures, such as (anti)microbial control and sanitation of feed and drinking water, respectively, as well as specific probiotics or short-chain fatty acids. Alternatively, or in addition thereto, higher-quality feed may be applied. The term “higher-quality feed” is to be understood as feed raw materials displaying a lower amount of antinutritional factors and/or nondigestable polymers being responsible for viscosity issues of the chime.

In case the farmer suspects an infection of birds with Salmonella and/or Campylobacter, these “soft measures” usually are triggered first. Only if there is an indication that the measure is not sufficiently effective, or if the Salmonella and/or Campylobacter load is initially too high, the farmer will, in accordance with the teaching of the present invention, switch to “medium measures”. Those “medium measures” include supplementary feeding of probiotic agents, praebiotic agents or botanicals and/or immunostimulation, feed supplementation by organic/fatty acids, bacteriophages and bacteriolytic enzymes and/or specific probiotic agents, used either alone or in combination of more than one of these “medium measures”. Further, if the “medium measures” are not sufficiently effective, or if the bacterial load is initially too high, the farmer will, in accordance with the teaching of the present invention, switch to “hard interventions”. The term “hard interventions” involves the specific treatment by administering a therapeutic agent, specific and non-specific antibiotic treatments and, at worst, culling of infected animals or culling of the whole flock.

The expression “enhancement of measures” is either to be understood as a transition from a soft measure to a medium measure or from a medium measure to a hard intervention; or as enhancing the intensity of a previously applied measure, e.g. by increasing the dosage or application frequency of a substance to be administered to the animals belonging to the animal flock.

By the above method, the occurrence of an unfavorable Salmonella and/or Campylobacter load can be determined and also quantified at a very early stage of infection, i.e. at a point in time where the animals are void of any abnormalities or symptoms.

Accordingly, the Salmonella and/or Campylobacter contamination can be determined—and also treated—at an early stage at which a veterinarian or a livestock manager applying conventional methods would not suspect the animal flock to be already contaminated by Salmonella and/or Campylobacter.

The outcome of such in-process measures and/or interventions then has to be controlled in the ongoing production process.

In one embodiment, the sample material of the test sample is a pooled sample material of an animal flock, preferably a poultry flock. The pooled sample material may be selected from the group consisting of dust samples, litter samples, liquid manure samples, fur samples, feather samples, skin samples, feed samples, bedding samples and samples of bodily excrements and solutions or suspensions thereof. Bodily excrements are urine, fecal or cecal excrements. Preferably, the sample material is feces.

The above-described methods are particularly suitable for monitoring the level of the Salmonella and/or Campylobacter load in an animal population over time. Thereby both, acute and relapsing phases of Salmonella and/or Campylobacter contaminations are detected reliably and efficiently.

In addition to the above, the present invention also pertains to a diagnostic kit comprising primers and optionally comprising probes for detecting a specific biomarker, wherein said biomarker comprises at least one polynucleotide being specific for the genus Salmonella and/or for the genus Campylobacter. In preferred embodiments, the biomarker comprises at least one polynucleotide being specific for Salmonella and/or Campylobacter species and/or sub-species and/or serovars thereof.

In one aspect the Salmonella is Salmonella enterica or Salmonella enterica ssp. enterica or its serovars Salmonella enteritidis, Salmonella typhimurium, Salmonella heidelberg, Salmonella kentucky, or any combinations thereof. In another aspect, the Campylobacter is Campylobacter jejuni, Campylobacter coli, Campylobacter lari, Campylobacter upsaliensis, or any combinations thereof. In a different aspect, the Salmonella is Salmonella enterica or Salmonella enterica ssp. enterica or its serovars Salmonella enteritidis, Salmonella typhimurium, Salmonella heidelberg, Salmonella kentucky, or any combinations thereof and the Campylobacter is Campylobacter jejuni, Campylobacter coli, Campylobacter lari, Campylobacter upsaliensis, or any combinations thereof.

Diagnostically relevant genes of Campylobacter species Campylobacter jejuni are, for example, the 23S rRNA gene, the 16S rRNA gene, the 5S rRNA gene, cdtA, cdtB, cdtC, gyrB, glyA, cadF, tuf, atpA, wla genes, cjaA, trpC, cja, cjo, hipO, flaA, fusA, lpxA, Cj0414, and mapA. In a particularly preferred embodiment, the Campylobacter jejuni marker gene is selected from lpxA, Cj0414 and mapA. The marker gene Cj0414 is of particular relevance.

Diagnostically relevant genes of Salmonella species Salmonella enterica ssp. enterica are, for example, ompf gene, inva gene, 23S rRNA gene, 16S rRNA gene, 5S rRNA gene, mutS, recA, arac gene, rpob gene, Salmolysin, mdh gene, tuf A,B genes, phop gene, gyrA gene, parc gene, invE gene and prgH. In a particularly preferred embodiment, the Salmonella enterica ssp. enterica marker gene is selected from the group invA, fliC, invE and hilA. The marker gene hilA is of particular relevance.

In a particularly preferred embodiment, the kit comprises

(i) at least one forward primer and at least one reverse primer to amplify the above polynucleotides, and
(ii) calibrators and controls

The kit may further comprise buffer solutions, such as PCR buffer; magnesia salts; deoxy nucleotide triphosphates (dNTPs), and a DNA polymerase. The kit may also include elements such as sample collection tubes, reagents to isolate the nucleic acids and/or instructions for its use.

Applications of the methods according to the invention are for example (i) monitoring the bacterial load/microbial contamination of an animal flock, (ii) controlling the effectiveness of anti-microbial actions and (iii) aiding in the evaluation of treatment efficacy for an animal population undergoing or contemplating treatment.

### BRIEF DESCRIPTION OF THE FIGURES

FIG. 1 (A) to (D) shows the growth curves received from qPCR data of (A) very high, (B) high, (C) medium and (D) low concentrated inoculum concentrations for untreated Salmonella cultures and cultures treated with Kanamycin, Sodium butyrate and Ecobiol (cell-free and cell-containing).

FIGS. 2 (A) and (B) depicts the growth rate (A) calculated for the Salmonella MTP assay and delta growth rate (B) in reference to the untreated control for the different inoculum concentrations (very high to low).

FIG. 3 (A) to (D) shows the growth curves received from qPCR data of (A) very high, (B) high, (C) medium and (D) low concentrated inoculum concentrations for untreated Campylobacter cultures and cultures treated with Kanamycin, Sodiumbutyrate and GutCare (cell-free).

FIGS. 4 (A) and (B) depicts the growth rate (A) calculated for the Campylobacter MTP assay and delta growth rate (B) in reference to the untreated control for the different inoculum concentrations (very high to low).

In the following, the invention is illustrated by non-limiting examples and exemplifying embodiments.

### Examples

The pathogens of interest Salmonella enterica subsp. enterica and Campylobacter jejuni were cultivated in microtiter plate scale in order to enable the simultaneous application of four different starting culture concentrations and treatments (Table 3). For analysis, samples for DNA extraction were taken after different times of cultivation and applied for qPCR enumeration of Salmonella enterica subsp. enterica and Campylobacter jejuni specific genes. As the qPCR assay is designed to record both viable and non-viable cells, an additional viability check was performed for AB treated cultures and the untreated controls.

TABLE 3
Experimental concept of the microtiter plate cultivation assay for
monitoring the effectiveness of treatments against Salmonella enterica
subsp. enterica and Campylobacter jejuni via qPCR

Inoculum concentration for MTP cultivation

| | | | | |
|---|---|---|---|---|
| Treatment | Very high | High | Medium | Low |

| | |
|---|---|
| Hard | Antibiotics: Kanamycin |
| Medium | Probiotics: Ecobiol/GutCare |
| Soft | Short chain fatty acids: Sodium butyrate |
| Control | Without treatment |

Sampling for qPCR

| | | | | |
|---|---|---|---|---|
| Organism | Very high | High | Medium | Low |

| | |
|---|---|
| Salmonella | 0, 2, 4, 7, 24 and 30 h |
| Campylobacter | 0, 7, 24 and 48 h |

| | Sampling for drop plate enumeration | | | |
|---|---|---|---|---|
| Treatment | Very high | High | Medium | Low |
| Hard | 0 and 6 h | | | 0 and 6 h |
| Control | 0 and 6 h | | | 0 and 6 h |

### Material and Methods

### Bacterial Strains

For Salmonella enterica subsp. enterica experiments, the DSMZ strain DSM-5569 was used. The inoculum culture was produced by an overnight cultivation in lysogeny broth+glucose (LBG) at 37° C. under aerobic conditions.

For Campylobacter jejuni experiments the DSMZ strain DSM-24306 was applied. The inoculum culture was produced by a first overnight cultivation on Columbia Blood Agar (CBA) followed by an overnight cultivation in Mueller-Hinton broth (MH). All cultivations took place under microaerophilic conditions (5% O₂, 10% CO₂, and 75% N₂) at 41° C.

### Microtiterplate Cultivation

The actual assay was carried out in microtiter plate format containing a total volume of 200 μl. Following media for the different treatment conditions were prepared according to Table 4. For the probiotic treatments (Ecobiol and GutCare), sterile filtrated supernatant of an overnight culture (=cell-free) or non-sterile filtrated, cell-containing supernatant of the same overnight culture was applied.

TABLE 4
Treatment substances and concentrations applied for microtiter
plate scale cultivation of S. enterica and C. jejuni.

| | Salmonella | | | Campylobacter | | |
|---|---|---|---|---|---|---|
| Measure | Substance | Concentration | | Substance | Concentration | |
| Soft | Butyrate | 25 | mM | Butyrate | 25 | mM |
| Medium | Ecobiol | 10% | (v/v) | GutCare | 10% | (v/v) |
| | (cell-free | | | (cell-free) | | |
| | or cell- | | | | | |
| | containing) | | | | | |
| Hard | Kanamycin | 50 | μl/ml | Kanamycin | 50 | μl/ml |

For C. jejuni all treatments were prepared in MH medium and for S. enterica in LBG respectively. Strains were cultivated under the conditions described in 2.1. Inoculated medium without any treatment served as an untreated control.

For each intervention and for the untreated control, following inoculum concentrations given in starting quantity of the Salmonella enterica subsp. enterica and Campylobacter jejuni specific target gene per ml culture were applied for the start of the assay:

TABLE 5
Starting concentrations of the freshly inoculated very high, high,
medium and low concentrated Salmonella and Campylobacter cultures
in starting quantity of the target gene per ml culture.

| Inoculum | Salmonella | Campylobacter |
|---|---|---|
| concentration | [SQ/ml culture] | [SQ/ml culture] |
| Very high | 1.88E+06 | 3.42E+07 |
| High | 5.50E+05 | 1.36E+07 |
| Medium | 1.48E+05 | 4.29E+06 |
| Low | 4.66E+04 | 2.63E+06 |

From each concentration and treatment, enough wells were prepared to extract DNA from three well replicates per time point and treatment. For Campylobacter, DNA was extracted after 0, 7, 24 and 48 h. For Salmonella, 0, 2, 4, 6, 24 and 30 h of cultivation were chosen.

### DNA Extraction

DNA extraction took place by transferring 100 μl of the culture into 900 μl lysis buffer. The sample was lysed by 20 min incubation at 70° C. and subsequent centrifugation at 3000 g for 5 min. 500 μl of the lysed sample were applied for magnetic bead-based semiautomatic DNA extraction. DNA eluates were used for further analysis

qPCR Detection

For enumeration of Salmonella or Campylobacter, primers and probes binding in conserved genetic regions, specific for each species were applied in qPCR assays with a total volume of 30 μl and a DNA eluate volume of 10 μl. Target genes and references are depicted in Table 6. Absolute quantification of targets was performed under consolidation of standard curves that were carried with each run. Using the standard curves, the starting quantity (SQ) of the specific target was calculated for each sample. Internal extraction controls were carried with every sample in order to ensure the validity of negative samples.

TABLE 6
Target genes and references for Salmonella enterica subspecies
enterica and Campylobacter jejuni qPCR detection

| | Target Gene | Protein | Reference Strain | GenBank | Location |
|---|---|---|---|---|---|
| Target Species | [Synonyms] | Description | Description | Accession No. | [bp] |
| Salmonella enterica | hilA | transcriptional | Salmonella enterica | NC_003197.2 | 3019846- |
| subspecies enterica | [iagA] | regulator | subspecies enterica | | 3021524 |
| serovars | | | serovar Typhimurium | | |
| | | | strain LT2, complete | | |
| | | | genome | | |
| Campylobacter jejuni | Cj0414 | Oxireductase | Campylobacter jejuni | NC_009839.1 | 380937- |
| | | subunit | subspecies jejuni | | 381665 |
| | | | NCTC 81116 | | |

### Calculation of Generation Time and Growth Rate

In order to calculate the growth rate of each culture during the exponential phase of growth following equation was applied:

$$Growth rate μ = \frac{log 10 ( {N}_{1} ) - log 1 C ( {N}_{0} )}{log 10 ( e ) * ( t - {t}_{0} )}$$

$$N = SQ \text{/} ml culture$$

To outline the effect of each treatment, the difference of growth rates between untreated and treated cultures was calculated for each starting inoculum concentration as following:

Delta μ=μ_{treated culture}−μ_{untreated control}

### Drop Plate Enumeration of Viable Cells

In order to evaluate the effect of bactericidic effect Kanamycin, drop plate tests were performed to determine the number of viable cells. Three dilutions of a ten-fold series of the high (dilutions 10⁻³, 10⁻⁴ and 10⁻⁵) and low (dilutions 10′, 10⁻² and 10⁻³) inoculated culture after 0 and 6 h were plated in triplicates on CBA (Campylobacter) or tryptic soy agar (TSA) (Salmonella) by dropping 10 μl on the agar. The plates were evaluated regarding growth at the distinct log dilutions after 24 h incubation (41° C. microaerophilic for Campylobacter, 37° C. aerobic for Salmonella).

### Results

### Salmonella

### Growth Curves

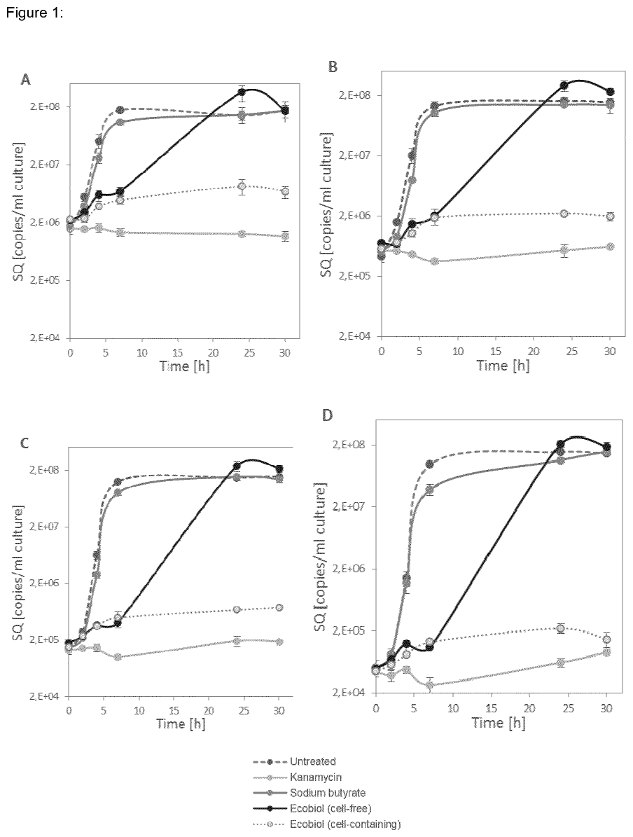

The growth curves in the form of target starting quantity (SQ) per ml culture for the four different starting density Salmonella DSM-5569 cultures with the respective treatments are depicted in FIG. 1.

While the untreated cultures as well as the sodium butyrate treated cultures show a strong exponential growth during the first 7 h for all inoculum concentrations, a delay of the exponential phase is observed for the Ecobiol (cell-free) treated cultures. For the treatments with Kanamycin and cell-containing Ecobiol, a clear growth inhibition can be seen for the full period of cultivation and all inoculum densities. Kanamycin leads to an over-all lower starting quantity of the target gene than cell-containing Ecobiol.

### Drop Plate Enumeration of Viable Cells

The not only inhibitory but also bactericidal effect of Kanamycin on S. enterica was proved using the agar drop assay in a separate test (Table 7). While similar amounts of viable cells were found both in Kanamycin treated and untreated culture (log 6-7 for the high concentrated inoculum, 5 for the low concentrated inoculum) at the start of cultivation, no growth is detected after 6 h of incubation in presence of Kanamycin. This results confirms that the qPCR assay detects both viable and dead cells, explaining the lack of a copy number decrease in the Kanamycin treated culture.

TABLE 7
Drop plate assay for evaluating viability of high and
low inoculated Salmonella cultures after 0 h of
untreated and Kanamycin treated cultivation and after
6 h of untreated and Kanamycin treated cultivation.

| | High concentrated | | Low concentrated | |
|---|---|---|---|---|
| Dilution | Untreated | Kanamycin | Untreated | Kanamycin |
| | After 0 h | | | |
| 10⁻³ | | | +++ | +++ |
| 10⁻⁴ | | | ++ | ++ |
| 10⁻⁵ | +++ | +++ | + | + |
| 10⁻⁶ | ++ | ++ | | |
| 10⁻⁷ | − | + | | |
| | After 6 h | | | |
| 10⁻³ | | | +++ | − |
| 10⁻⁴ | | | +++ | − |
| 10⁻⁵ | +++ | − | +++ | − |
| 10⁻⁶ | +++ | − | | |
| 10⁻⁷ | +++ | − | | |

+++ bacterial lawn
++ few colonies
+ single colonies
− no growth observable
not evaluated

### Growth Rates

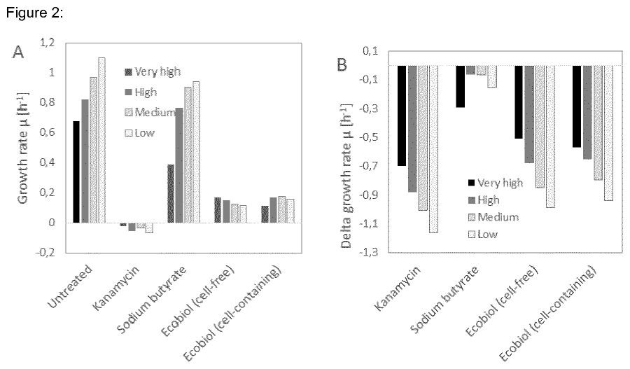

The growth rate calculated for the Salmonella enterica microtiter plate assay was evaluated for the cultivation period of 0 to 7 h and is depicted in FIG. 2A. The untreated controls grew with rates ranging from 0.6 to 1.1 with an increasing growth rate with decreasing inoculum concentration. The biggest effect on the growth rate is observed for the Kanamycin treatment were negative rates were calculated for all cultures. For sodium butyrate comparably high growth rates ranging from 0.4 to 0.9 were recorded. Ecobiol shows similar growth rate reducing effects for both constitutions (cell-free and cell-containing), as the significant difference between both treatments was only observable from 24 h ongoing.

The difference of the growth rates (delta) from untreated control and each treatment was calculated in order to evaluate the effect of the treatment on the growth rate in relation to the inoculum concentration (FIG. 2B). The strongest effect is found for the low concentrated starting cultures for the Kanamycin and the both Ecobiol treatments with decreasing deltas for the increasing inoculum concentrations. Only for sodium butyrate the biggest effect is found for the high concentrated inoculum, however the effect is rather low for all concentrations.

### Campylobacter

### Growth Curves

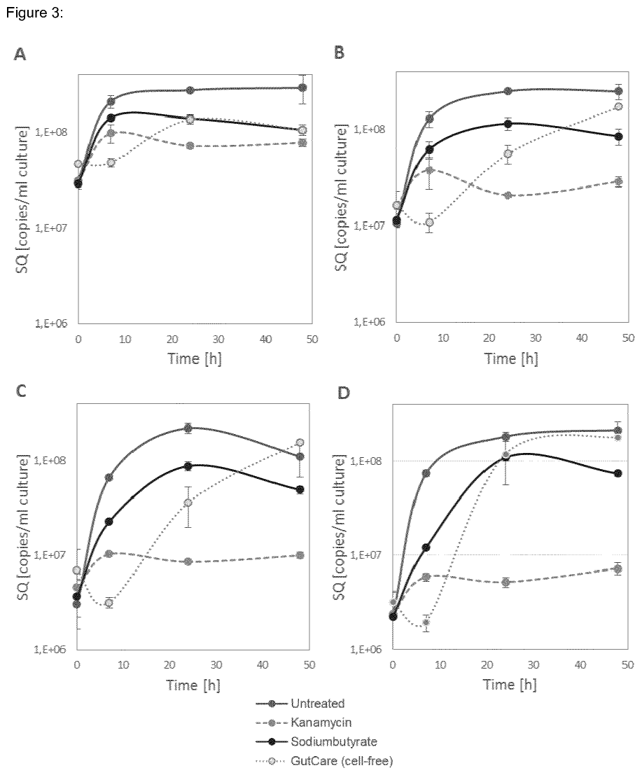

The growth curves in the form of target starting quantity (SQ) per ml culture for the four different starting density Campylobacter DSM-24306 cultures with the respective treatments are depicted in FIG. 3. Over all, the different treatments lead to similar effects in all inoculum concentrations. Likewise to Salmonella, an exponential growth until at least 7 h of cultivation was observed for the untreated control. Sodium butyrate shows an inhibitory effect on Campylobacter jejuni: although exponential growth was observed, significantly lower plateau SQ is reached. For the treatment with cell-free GutCare not only a delay in growth start, but also a reduction in SQ can be seen during the first 7 h of cultivation for all inoculum concentrations. However after 48 h, Campylobacter reaches an end concentration similar to the untreated control in these cultures. Kanamycin leads to an inhibition of growth after a weak increasement of SQ during the first 7 h.

### Drop Plate Enumeration of Viable Cells

The not only inhibitory but also bactericidal effect of Kanamycin on C. jejuni was proved using the agar drop assay (Table 8) in a separate test. While similar amounts of viable cells were found both in Kanamycin treated and untreated culture (log 6-7 for the high concentrated inoculum, 5 for the low concentrated inoculum) at the start of cultivation, no growth is detected after 6 h of incubation in presence of Kanamycin. This results confirms that the qPCR assay detects both viable and dead cells, explaining the lack of a copy number decrease in the Kanamycin treated culture.

TABLE 8
Drop plate assay for evaluating viability of high and
low inoculated Campylobacter cultures after 0 h of
untreated and Kanamycin treated cultivation and after
6 h of untreated and Kanamycin treated cultivation.

| | High concentrated | | Low concentrated | |
|---|---|---|---|---|
| Dilution | Untreated | Kanamycin | Untreated | Kanamycin |
| | After 0 h | | | |
| 10⁻³ | | | +++ | ++ |
| 10⁻⁴ | | | ++ | ++ |
| 10⁻⁵ | ++ | ++ | + | + |
| 10⁻⁶ | ++ | + | | |
| 10⁻⁷ | + | − | | |
| | After 6 h | | | |
| 10⁻³ | | | +++ | − |
| 10⁻⁴ | | | +++ | − |
| 10⁻⁵ | +++ | − | ++ | − |
| 10⁻⁶ | ++ | − | | |
| 10⁻⁷ | + | − | | |

+++ bacterial lawn
++ few colonies
+ single colonies
− no growth observable
not evaluated

### Growth Rates

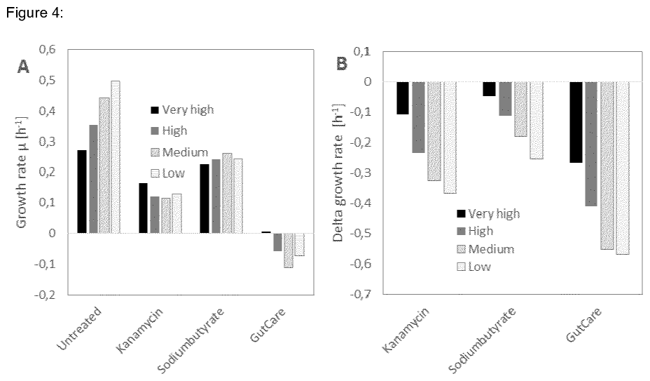

The growth rate for the Campylobacter jejuni microtiter plate assay was evaluated for the cultivation period of 0 to 7 h and is depicted in FIG. 4A. The untreated controls grew with rates ranging from 0.25 to 0.5 and showed an increasing growth rate with decreasing inoculum concentration. The biggest effect on the growth rate is observed for the GutCare treatment were negative rates were calculated for all cultures. For sodium butyrate growth rates ranging around 0.24 were received and 0.13 for Kanamycin respectively.

The difference of the growth rates (delta) from untreated control and each treatment was calculated in order to evaluate the effect of the treatment on the growth rate in relation to the inoculum concentration (FIG. 4B). The biggest effect is found for the low concentrated starting cultures for all treatments with decreasing deltas for the increasing inoculum concentrations.

To summarize, the qPCR assays are able to quantify specific loads of Salmonella and Campylobacter and to monitor the effect of a treatment on the propagation of these pathogens. Also, differences in treatment efficiency are detected, which enables the adjustment of treatments appropriate to the bacterial load and growth rate. Accordingly, the sufficiency of soft measures for low pathogen loads and growth rates can be monitored and the switch to a harder intervention can be triggered in case of high loads and increasing propagation.

1-15. (canceled)

16. An in vitro method for determining the necessity of initiating or enhancing measures against the propagation of Salmonella and/or against the propagation of Campylobacter in an animal flock, comprising:
i) determining the amount of at least one marker gene that is specific for Salmonella or Campylobacter in a test sample; and
ii) comparing the amount of said at least one marker determined in the test sample with a control sample;
wherein:
the test sample is pooled fecal sample material of the animal flock;
the at least one marker gene specific for Salmonella is hilA, and the at least one marker gene specific for Campylobacter is Cj0414;
an increase in the amount of said at least one marker gene specific for Salmonella or Campylobacter in the test sample vs. the control sample by at least a factor of two, indicates the necessity of initiating or enhancing measures against the propagation of Salmonella and/or against the propagation Campylobacter.

17. The method of claim 16, wherein the control sample is a sample of the animal flock obtained previously.

18. The method of claim 16, wherein the control sample is a negative control sample.

19. The method of claim 16, wherein the marker genes are quantified via qPCR.

20. The method of claim 16, wherein the animal in said animal flock is poultry.

21. The method of claim 16, wherein after an initial determination, the amount of said at least one specific biomarker is monitored in test samples collected and analyzed in a weekly, daily or hourly manner.

22. The method of claim 16, wherein measures against Salmonella and/or against Campylobacter comprise feeding zootechnical feed additives or therapeutic agents.

23. A method for monitoring the level of the Salmonella and/or Campylobacter load in an animal population, comprising repeatedly performing the method of claim 16 over time.

24. The method of claim 23, wherein the marker genes are quantified via qPCR.

25. The method of claim 24, wherein the animal in said animal flock is poultry.

26. The method of claim 25, wherein after an initial determination, the amount of said at least one specific biomarker is monitored in test samples collected and analyzed in a weekly, daily or hourly manner.

27. The method of claim 26, wherein measures against Salmonella and/or against Campylobacter comprise feeding zootechnical feed additives or therapeutic agents.

28. The method of claim 17, wherein the marker genes are quantified via qPCR.

29. The method of claim 28, wherein the animal in said animal flock is poultry.

30. The method of claim 29, wherein after an initial determination, the amount of said at least one specific biomarker is monitored in test samples collected and analyzed in a weekly, daily or hourly manner.

31. The method of claim 16, wherein measures against Salmonella and/or against Campylobacter comprise feeding zootechnical feed additives, or therapeutic agents.

32. The method of claim 31, wherein the marker genes are quantified via qPCR.

33. The method of claim 32, wherein the animal in said animal flock is poultry.

34. The method of claim 33, wherein after an initial determination, the amount of said at least one specific biomarker is monitored in test samples collected and analyzed in a weekly, daily or hourly manner.

35. The method of claim 34, wherein measures against Salmonella and/or against Campylobacter comprise feeding therapeutic agents.
